(19) United States
(12) Patent Application Publication
(10) Pub. No.: US 2010/0278921 A1
(43) Pub. Date: Nov. 4, 2010

(54) SOLID ORAL FORMULATION OF ABT-263

(76) Inventors: Cristina M. Fischer, Wadsworth, IL (US); Rajeev Gokhale, Libertyville, IL (US); Katherine Heemstra, Chicago, IL (US); David Hill, Gurnee, IL (US); Kennan Marsh, Lake Forest, IL (US); Eric A. Schmitt, Libertyville, IL (US); Yi Shi, Libertyville, IL (US); Ping Tong, Libertyville, IL (US); Deliang Zhou, Vernon Hills, IL (US)

Correspondence Address: Harness Dickey & Pierce, PLC, 7700 Bonhomme, Suite 400, Clayton, MO, 63105, US

(21) Appl. No.: 12/770,205

(22) Filed: Apr. 29, 2010

Related U.S. Application Data
(60) Provisional application No. 61/174,318, filed on Apr. 30, 2009.

(51) Int. Cl. A61K 31/5377; A61K 9/14; A61P 35/00

(52) U.S. Cl. 424/489; 514/235.8

(57) ABSTRACT

An orally deliverable pharmaceutical composition comprises (a) a pharmaceutically acceptable acid addition salt of ABT-263 in solid particulate form, and (b) a plurality of pharmaceutically acceptable excipients including at least a solid diluent and a solid disintegrant; wherein the salt is formed from more than one equivalent of acid per equivalent of ABT-263. The composition is suitable for oral administration to a subject in need thereof for treatment of a disease characterized by overexpression of one or more anti-apoptotic Bcl-2 family proteins, for example cancer.

### CROSS-REFERENCE TO RELATED APPLICATIONS

This application claims priority benefit of U.S. Provisional Application Ser. No. 61/174,318 filed on Apr. 30, 2009.

Cross-reference is made to the following co-filed U.S. applications containing subject matter related to the present application: Ser. No. 12/______ titled “Salt of ABT-263 and solid-state forms thereof”, which claims priority benefit of U.S. provisional application Ser. No. 61/174,274 filed on Apr. 30, 2009; and Ser. No. 12/______ titled “Formulation for oral administration of apoptosis promoter”, which claims priority benefit of above-referenced U.S. provisional application Ser. No. 61/174,318, as well as Ser. No. 61/174,299 filed on Apr. 30, 2009, Ser. No. 61/174,318 filed on Apr. 30, 2009, Ser. No. 61/185,105 filed on Jun. 8, 2009, Ser. No. 61/185,130 filed on Jun. 8, 2009, Ser. No. 61/218,281 filed on Jun. 18, 2009, Ser. No. 61/289,254 filed on Dec. 22, 2009, and Ser. No. 61/289,289 filed on Dec. 22, 2009.

The entire disclosure of each of the above applications is incorporated herein by reference.

### FIELD OF THE INVENTION

The present invention relates to pharmaceutical compositions comprising the apoptosis-promoting agent ABT-263, and to methods of use thereof for treating diseases characterized by overexpression of anti-apoptotic Bcl-2 family proteins. More particularly the invention relates to such compositions in solid form, for example as tablets or capsules, and to oral dosage regimens for administration of such a composition to a subject in need thereof.

### BACKGROUND OF THE INVENTION

Evasion of apoptosis is a hallmark of cancer (Hanahan & Weinberg (2000) Cell 100:57-70). Cancer cells must overcome a continual bombardment by cellular stresses such as DNA damage, oncogene activation, aberrant cell cycle progression and harsh microenvironments that would cause normal cells to undergo apoptosis. One of the primary means by which cancer cells evade apoptosis is by up-regulation of anti-apoptotic proteins of the Bcl-2 family.

Compounds that occupy the BH3 binding groove of Bcl-2 proteins have been described, for example by Bruncko et al. (2007) J. Med. Chem. 50:641-662. These compounds have included N-(4-(4-((4′-chloro-(1,1′-biphenyl)-2-yl)methyl)piperazin-1-yl) benzoyl)-4-(((1R)-3-(dimethylamino)-1-((phenylsulfanyl)methyl)propyl)amino)-3-nitrobenzene-sulfonamide, otherwise known as ABT-737, which has the formula:

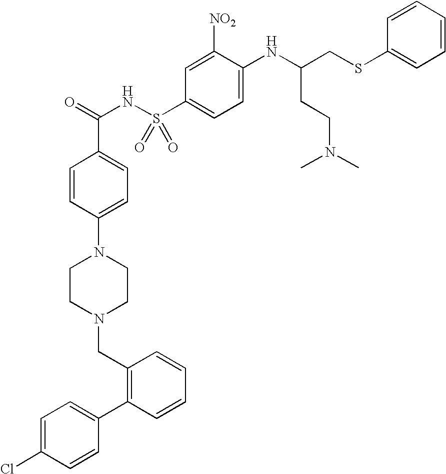

ABT-737 binds with high affinity (<1 nM) to proteins of the Bcl-2 family (specifically Bcl-2, Bcl-X_{L} and Bcl-w). It exhibits single-agent activity against small-cell lung cancer (SCLC) and lymphoid malignancies, and potentiates pro-apoptotic effects of other chemotherapeutic agents. ABT-737 and related compounds, and methods to make such compounds, are disclosed in U.S. Patent Application Publication No. 2007/0072860 of Bruncko et al.

More recently, a further series of compounds has been identified having high binding affinity to Bcl-2 family proteins. These compounds, and methods to make them, are disclosed in U.S. Patent Application Publication No. 2007/0027135 of Bruncko et al. (herein “the '135 publication”), incorporated by reference herein in its entirety, and can be seen from their formula to be structurally related to ABT-737.

One compound, identified as “Example 1” in the '135 publication, is N-(4-(4-((2-(4-chlorophenyl)-5,5-dimethyl-1-cyclohex-1-en-1-yl)methyl)piperazin-1-yl)benzoyl)-4-4-(((1R)-3-(morpholin-4-yl)-1-((phenylsulfanyl)methyl)propyl)amino-3-((trifluoromethyl)sulfonyl) benzenesulfonamide, otherwise known as ABT-263. This compound has a molecular weight of 974.6 g/mol and has the formula:

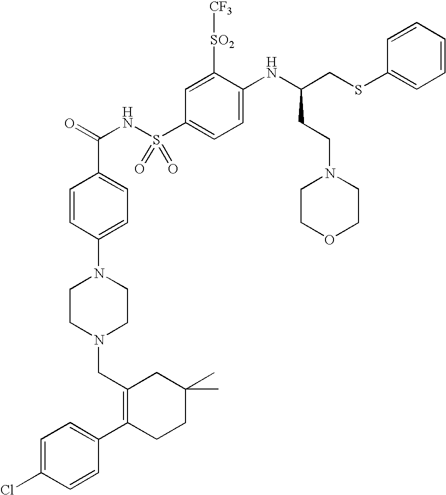

The '135 publication states that while inhibitors of Bcl-2 family proteins previously known may have either potent cellular efficacy or high systemic exposure after oral administration, they do not possess both properties. A typical measure of cellular efficacy of a compound is the concentration eliciting 50% cellular effect (EC₅₀). A typical measure of systemic exposure after oral administration of a compound is the area under the curve (AUC) resulting from graphing plasma concentration of the compound versus time from oral administration. Previously known compounds, it is stated in the '135 publication, have a low AUC/EC₅₀ ratio, meaning that they are not orally efficacious. By contrast, compounds provided therein are stated to demonstrate enhanced properties with respect to cellular efficacy and systemic exposure after oral administration, resulting in a AUC/EC₅₀ ratio significantly higher than that of previously known compounds.

ABT-263 binds with high affinity (<1 nM) to Bcl-2 and Bcl-X_{L} and is believed to have similarly high affinity for Bcl-w. Its AUC/EC₅₀ ratio is reported in the '135 publication as 56, more than an order of magnitude greater than that reported for ABT-737 (4.5). For determination of AUC according to the '135 publication, each compound was administered to rats in a single 5 mg/kg dose by oral gavage as a 2 mg/ml solution in a vehicle of 10% DMSO (dimethyl sulfoxide) in PEG-400 (polyethylene glycol of average molecular weight about 400).

Oral bioavailability (as expressed, for example, by AUC after oral administration as a percentage of AUC after intravenous administration) is not reported in the '135 publication, but can be concluded therefrom to be substantially greater for ABT-263 than for ABT-737. It is not known, however, whether ABT-263 can be formulated as a convenient solid tablet or capsule formulation, or if when so formulated its oral bioavailability will at least measure up to that of the solution in PEG-400/DMSO 10:1 disclosed in the '135 publication.

A particular type of disease for which improved therapies are needed is non-Hodgkin's lymphoma (NHL). NHL is the sixth most prevalent type of new cancer in the U.S. and occurs primarily in patients 60-70 years of age. NHL is not a single disease but a family of related diseases, which are classified on the basis of several characteristics including clinical attributes and histology.

One method of classification places different histological subtypes into two major categories based on natural history of the disease, i.e., whether the disease is indolent or aggressive. In general, indolent subtypes grow slowly and are generally incurable, whereas aggressive subtypes grow rapidly and are potentially curable. Follicular lymphomas are the most common indolent subtype, and diffuse large-cell lymphomas constitute the most common aggressive subtype. The oncoprotein Bcl-2 was originally described in non-Hodgkin's B-cell lymphoma.

Treatment of follicular lymphoma typically consists of biologically-based or combination chemotherapy. Combination therapy with rituximab, cyclophosphamide, doxorubicin, vincristine and prednisone (R-CHOP) is routinely used, as is combination therapy with rituximab, cyclophosphamide, vincristine and prednisone (RCVP). Single-agent therapy with rituximab (targeting CD20, a phosphoprotein uniformly expressed on the surface of B-cells) or fludarabine is also used. Addition of rituximab to chemotherapy regimens can provide improved response rate and increased progression-free survival.

Radioimmunotherapy agents, high-dose chemotherapy and stem cell transplants can be used to treat refractory or relapsed non-Hodgkin's lymphoma. Currently, there is not an approved treatment regimen that produces a cure, and current guidelines recommend that patients be treated in the context of a clinical trial, even in a first-line setting.

First-line treatment of patients with aggressive large B-cell lymphoma typically consists of rituximab, cyclophosphamide, doxorubicin, vincristine and prednisone (R-CHOP), or dose-adjusted etoposide, prednisone, vincristine, cyclophosphamide, doxorubicin and rituximab (DA-EPOCH-R).

Most lymphomas respond initially to any one of these therapies, but tumors typically recur and eventually become refractory. As the number of regimens patients receive increases, the more chemotherapy-resistant the disease becomes. Average response to first-line therapy is approximately 75%, 60% to second-line, 50% to third-line, and about 35-40% to fourth-line therapy. Response rates approaching 20% with a single agent in a multiple relapsed setting are considered positive and warrant further study.

Current chemotherapeutic agents elicit their antitumor response by inducing apoptosis through a variety of mechanisms. However, many tumors ultimately become resistant to these agents. Bcl-2 and Bcl-X_{L} have been shown to confer chemotherapy resistance in short-term survival assays in vitro and, more recently, in vivo. This suggests that if improved therapies aimed at suppressing the function of Bcl-2 and Bcl-X_{L} can be developed, such chemotherapy-resistance could be successfully overcome.

Apoptosis-promoting drugs that target Bcl-2 family proteins such as Bcl-2 and Bcl-X_{L} are best administered according to a regimen that provides continual, for example daily, replenishment of the plasma concentration, to maintain the concentration in a therapeutically effective range. This can be achieved by daily parenteral, e.g., intravenous (i.v.) or intraperitoneal (i.p.) administration. However, daily parenteral administration is often not practical in a clinical setting, particularly for outpatients. To enhance clinical utility of an apoptosis-promoting agent, for example as a chemotherapeutic in cancer patients, a dosage form with acceptable oral bioavailability would be highly desirable. Such a dosage form, and a regimen for oral administration thereof, would represent an important advance in treatment of many types of cancer, including non-Hodgkin's lymphoma, and would more readily enable combination therapies with other chemotherapeutics.

### SUMMARY OF THE INVENTION

It has been found that oral bioavailability of the lead Bcl-2 protein family inhibitor ABT-737 is not substantially affected by the carrier system in which it is formulated. Despite this discouraging result, the present inventors have continued the search for a Bcl-2 protein family inhibitory composition and have discovered that the related compound ABT-263, when prepared as a bis-acid addition salt, can be formulated as a solid mix with excipients suitable for tableting or encapsulating, at a drug loading much higher than the 2 mg/ml (about 0.2%) solution reported in the '135 publication. Furthermore, they have discovered that such a solid formulation exhibits oral bioavailability that is at least comparable to, and in some cases superior to, that of the 2 mg/ml solution reported in the '135 publication.

There is accordingly provided an orally deliverable pharmaceutical composition comprising (a) a pharmaceutically acceptable acid addition salt of N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino-3-((trifluoromethyl)sulfonyl)benzenesulfonamide (ABT-263) in solid particulate form, and (b) a plurality of pharmaceutically acceptable excipients including at least a solid diluent and a solid disintegrant; wherein the salt is formed from more than one, for example two, equivalents of acid per equivalent of ABT-263. An example of such a salt is ABT-263 bis-hydrochloride salt (ABT-263 bis-HCl), having the formula

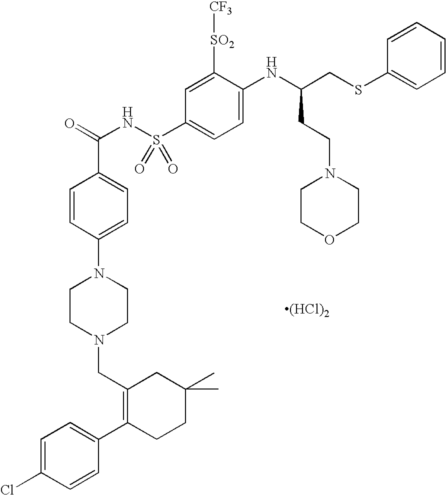

In various embodiments the composition comprises a solid granulate as can be prepared, for example, by dry or wet granulation processes, or a compressed powder as can be prepared, for example, by a direct compression process. Such a granulate or compressed powder can be formed as discrete oral dosage forms such as tablets or capsules.

There is further provided a method for treating a disease characterized by apoptotic dysfunction and/or overexpression of an anti-apoptotic Bcl-2 family protein, comprising orally administering to a subject having the disease a therapeutically effective amount of a composition as described immediately above. Examples of such a disease include many neoplastic diseases including cancers. A specific illustrative type of cancer that can be treated according to the present method is non-Hodgkin's lymphoma. Another specific illustrative type of cancer that can be treated according to the present method is chronic lymphocytic leukemia. Yet another specific illustrative type of cancer that can be treated according to the present method is acute lymphocytic leukemia, for example in a pediatric patient.

There is still further provided a method for maintaining in bloodstream of a human cancer patient, for example a patient having non-Hodgkin's lymphoma, chronic lymphocytic leukemia or acute lymphocytic leukemia, a therapeutically effective plasma concentration of ABT-263 and/or one or more metabolites thereof, comprising administering to the subject a pharmaceutical composition as described above, in a dosage amount equivalent to about 50 to about 500 mg ABT-263 per day, at an average dosage interval of about 3 hours to about 7 days.

Additional embodiments of the invention, including more particular aspects of those provided above, will be found in, or will be evident from, the detailed description that follows.

### DETAILED DESCRIPTION

A composition of the invention is “orally deliverable”, i.e., adapted for oral administration. The terms “oral administration” and “orally administered” herein refer to administration to a subject per os (p.o.), that is, administration wherein the composition is immediately swallowed, for example with the aid of a suitable volume of water or other potable liquid. “Oral administration” is distinguished herein from intraoral administration, e.g., sublingual or buccal administration or topical administration to intraoral tissues such as periodontal tissues, that does not involve immediate swallowing of the composition.

ABT-263 and its salts useful herein have very low solubility in water, generally less than about 10 μg/ml. It will be recognized that aqueous solubility of many compounds is pH dependent; in the case of such compounds the solubility of interest herein is at a physiologically relevant pH, for example a pH of about 1 to about 8. Illustratively, ABT-263 has a solubility in water at pH 2 (a pH at which the molecule is overwhelmingly protonated at least at two sites) of less than 4 μg/ml.

In free base form, for example as prepared according to Example 1 of the '135 publication, ABT-263 is an amorphous or glassy solid that is not well suited to preparation of solid dosage forms as desired herein. Certain salts of ABT-263 are more readily prepared in crystalline form, and consequently provide a active pharmaceutical ingredient (API) that is more suited to downstream formulation.

ABT-263 can form acid addition salts, basic addition salts or zwitterions. Acid addition salts of interest herein are those derived from reaction of ABT-263 free base with an acid. For example, salts including the acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifluoroacetate, para-toluenesulfonate and undecanoate salts of ABT-263 can be used in a composition of the invention.

ABT-263 has at least two protonatable nitrogen atoms and is consequently capable of forming acid addition salts with more than one, for example about 1.2 to about 2, about 1.5 to about 2 or about 1.8 to about 2, equivalents of acid per equivalent of the compound. Illustratively, bis-salts can be formed including any of those listed above, for example, bis-hydrochloride (bis-HCl) and bis-hydrobromide (bis-HBr) salts.

ABT-263 bis-HCl, which has a molecular weight of 1047.5 g/mol, can be prepared by a variety of processes, for example a process that can be outlined as follows.

ABT-263 free base is prepared, illustratively as described in Example 1 of above-cited U.S. Patent Application Publication No. 2007/0027135, the entire disclosure of which is incorporated by reference herein. A suitable weight of ABT-263 free base is dissolved in ethyl acetate. A solution of hydrochloric acid in ethanol (for example about 4.3 kg HCl in 80 g EtOH) is added to the ABT-263 solution in an amount providing at least 2 mol HCl per mol ABT-263 and sufficient EtOH (at least about 20 vol) for crystallization of the resulting ABT-263 bis-HCl salt. The solution is heated to about 45° C. with stirring and seeds are added as a slurry in EtOH. After about 6 hours, the resulting slurry is cooled to about 20° C. over about 1 hour and is mixed at that temperature for about 36 hours. The slurry is filtered to recover a crystalline solid, which is an ethanol solvate of ABT-263 bis-HCl. Drying of this solid under vacuum and nitrogen with mild agitation for about 8 days yields white desolvated ABT-263 bis-HCl crystals. This material is suitable for preparation of an ABT-263 bis-HCl formulation of the present invention.

The term “free base” is used for convenience herein to refer to the parent compound, while recognizing that the parent compound is, strictly speaking, zwitterionic and thus does not always behave as a true base.

Without being bound by theory, it is believed that the therapeutic efficacy of ABT-263 is due at least in part to its ability to bind to a Bcl-2 family protein such as Bcl-2, Bcl-X_{L} or Bcl-w in a way that inhibits the anti-apoptotic action of the protein, for example by occupying the BH3 binding groove of the protein.

ABT-263 in salt form as described herein is present in a composition of the present invention in an amount that can be therapeutically effective when the composition is administered to a subject in need thereof according to an appropriate regimen. Dosage amounts are expressed herein as free base equivalent amounts unless the context requires otherwise. Typically, a unit dose (the amount administered at a single time), which can be administered at an appropriate frequency, e.g., twice daily to once weekly, is about 10 to about 1,000 mg. Where frequency of administration is once daily (q.d.), unit dose and daily dose are the same thing. Illustratively, the unit dose of ABT-263 in a composition of the invention can be about 25 to about 1,000 mg, more typically about 50 to about 500 mg, for example about 50, about 100, about 150, about 200, about 250, about 300, about 350, about 400, about 450 or about 500 mg. Where the composition is prepared as a discrete dosage form such as a tablet or capsule, a unit dose can be deliverable in a single dosage form or a small plurality of dosage forms, most typically 1 to about 10 dosage forms.

The higher the unit dose, the more desirable it becomes to select excipients that permit a relatively high loading of the ABT-263 salt in the formulation. Typically, the concentration of ABT-263 in a formulation of the invention is at least about 1%, e.g., about 1% to about 50%, by weight, but lower and higher concentrations can be acceptable or achievable in specific cases. Illustratively, the ABT-263 concentration in various embodiments is at least about 2%, e.g., about 2% to about 40%, by weight, for example about 5%, about 10%, about 15%, about 20%, about 25% or about 30% by weight of the formulation.

The API, in this case an ABT-263 salt such as ABT bis-HCl, is present in the composition in solid particulate form. Particle size of the API is not narrowly critical, though results suggest that reduction in particle size can improve bioavailability. In compositions of the invention, the D₉₀ particle size (90% by volume of the API particles in their longest dimension are smaller than this) is typically about 2.5 to about 50 μm, for example about 3 to about 30 μm. API in the upper part of this D₉₀ range is typically unmilled. Reduction in particle size to the lower part of the D₉₀ range is achievable, for example, by pin-milling or jet-milling. In one embodiment, unmilled API having a D₉₀ of about 20 to about 30 μm is used. In another embodiment, pin-milled or jet-milled API having a D₉₀ of about 3 to about 10 μm is used. In still other embodiments, API of intermediate D₉₀, for example about 10 to about 20 μm, is used.

A composition of the invention comprises, in addition to the API, a plurality of pharmaceutically acceptable excipients including at least one or more solid diluents and one or more solid disintegrants. Optionally, the excipients further include one or more binding agents, wetting agents and/or antifrictional agents (lubricants, anti-adherents and/or glidants). Many excipients have two or more functions in a pharmaceutical composition. Characterization herein of a particular excipient as having a certain function, e.g., diluent, disintegrant, binding agent, etc., should not be read as limiting to that function. Further information on excipients can be found in standard reference works such as Handbook of Pharmaceutical Excipients, 3rd ed. (Kibbe, ed. (2000), Washington: American Pharmaceutical Association).

Suitable diluents illustratively include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents such as compressible sugar, confectioner's sugar and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dibasic calcium phosphate dihydrate, monobasic calcium sulfate monohydrate, calcium sulfate and granular calcium lactate trihydrate; magnesium carbonate; magnesium oxide; bentonite; kaolin; sodium chloride; and the like. Such diluents, if present, typically constitute in total about 5% to about 95%, for example about 20% to about 90%, or about 50% to about 85%, by weight of the composition. The diluent or diluents selected preferably exhibit suitable flow properties and, where tablets are desired, compressibility.

Microcrystalline cellulose and silicified microcrystalline cellulose are particularly useful diluents, and are optionally used in combination with a water-soluble diluent such as mannitol. Illustratively, a suitable weight ratio of microcrystalline cellulose or silicified microcrystalline cellulose to mannitol is about 10:1 to about 1:1, but ratios outside this range can be useful in particular circumstances.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. One or more disintegrants, if present, typically constitute in total about 0.2% to about 30%, for example about 0.5% to about 20%, or about 1% to about 10%, by weight of the composition.

Sodium starch glycolate is a particularly useful disintegrant, and typically constitutes in total about 1% to about 20%, for example about 2% to about 15%, or about 5% to about 10%, by weight of the composition.

Binding agents or adhesives are useful excipients, particularly where the composition is in the form of a tablet. Such binding agents and adhesives should impart sufficient cohesion to the blend being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; glucose; polydextrose; starch including pregelatinized starch; gelatin; modified celluloses including methylcellulose, carmellose sodium, hydroxypropylmethylcellulose (HPMC), hydroxypropylcellulose, hydroxyethylcellulose and ethylcellulose; dextrins including maltodextrin; zein; alginic acid and salts of alginic acid, for example sodium alginate; magnesium aluminum silicate; bentonite; polyethylene glycol (PEG); polyethylene oxide; guar gum; polysaccharide acids; polyvinylpyrrolidone (povidone or PVP), for example povidone K-15, K-30 and K-29/32; polyacrylic acids (carbomers); polymethacrylates; and the like. One or more binding agents and/or adhesives, if present, typically constitute in total about 0.5% to about 25%, for example about 1% to about 15%, or about 1.5% to about 10%, by weight of the composition.

Povidone and hydroxypropylcellulose, either individually or in combination, are particularly useful binding agents for tablet formulations, and, if present, typically constitute about 0.5% to about 15%, for example about 1% to about 10%, or about 2% to about 8%, by weight of the composition.

Wetting agents, if present, are normally selected to maintain the drug in close association with water, a condition that can improve bioavailability of the composition. Non-limiting examples of surfactants that can be used as wetting agents include, either individually or in combination, quaternary ammonium compounds, for example benzalkonium chloride, benzethonium chloride and cetylpyridinium chloride; dioctyl sodium sulfosuccinate; polyoxyethylene alkylphenyl ethers, for example nonoxynol 9, nonoxynol 10 and octoxynol 9; poloxamers (polyoxyethylene and polyoxypropylene block copolymers); polyoxyethylene fatty acid glycerides and oils, for example polyoxyethylene (8) caprylic/capric mono- and diglycerides, polyoxyethylene (35) castor oil and polyoxyethylene (40) hydrogenated castor oil; polyoxyethylene alkyl ethers, for example ceteth-10, laureth-4, laureth-23, oleth-2, oleth-10, oleth-20, steareth-2, steareth-10, steareth-20, steareth-100 and polyoxyethylene (20) cetostearyl ether; polyoxyethylene fatty acid esters, for example polyoxyethylene (20) stearate, polyoxyethylene (40) stearate and polyoxyethylene (100) stearate; sorbitan esters, for example sorbitan monolaurate, sorbitan monooleate, sorbitan monopalmitate and sorbitan monostearate; polyoxyethylene sorbitan esters, for example polysorbate 20 and polysorbate 80; propylene glycol fatty acid esters, for example propylene glycol laurate; sodium lauryl sulfate; fatty acids and salts thereof, for example oleic acid, sodium oleate and triethanolamine oleate; glyceryl fatty acid esters, for example glyceryl monooleate, glyceryl monostearate and glyceryl palmitostearate; α-tocopherol polyethylene glycol (1000) succinate (TPGS); tyloxapol; and the like. One or more wetting agents, if present, typically constitute in total about 0.1% to about 15%, for example about 0.2% to about 10%, or about 0.5% to about 7%, by weight of the composition.

Nonionic surfactants, more particularly poloxamers, are examples of wetting agents that can be useful herein. Illustratively, a poloxamer such as Pluronic™ F127, if present, can constitute about 0.1% to about 10%, for example about 0.2% to about 7%, or about 0.5% to about 5%, by weight of the composition.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. One or more lubricants, if present, typically constitute in total about 0.05% to about 10%, for example about 0.1% to about 5%, or about 0.2% to about 2%, by weight of the composition. Sodium stearyl fumarate is a particularly useful lubricant.

Anti-adherents reduce sticking of a tablet formulation to equipment surfaces. Suitable anti-adherents include, either individually or in combination, talc, colloidal silicon dioxide, starch, DL-leucine, sodium lauryl sulfate and metallic stearates. One or more anti-adherents, if present, typically constitute in total about 0.05% to about 10%, for example about 0.1% to about 7%, or about 0.2% to about 5%, by weight of the composition. Colloidal silicon dioxide is a particularly useful anti-adherent.

Glidants improve flow properties and reduce static in a tableting mixture. Suitable glidants include, either individually or in combination, colloidal silicon dioxide, starch, powdered cellulose, sodium lauryl sulfate, magnesium trisilicate and metallic stearates. One or more glidants, if present, typically constitute in total about 0.05% to about 10%, for example about 0.1% to about 7%, or about 0.2% to about 5%, by weight of the composition. Colloidal silicon dioxide is a particularly useful glidant.

Other excipients such as buffering agents, stabilizers, antioxidants, antimicrobials, colorants, flavors and sweeteners are known in the pharmaceutical art and can be used in compositions of the present invention. Tablets can be uncoated or can comprise a core that is coated, for example with a nonfunctional film or a release-modifying or enteric coating. Capsules can have hard or soft shells comprising, for example, gelatin (in the form of hard gelatin capsules or soft elastic gelatin capsules), starch, carrageenan and/or HPMC, optionally together with one or more plasticizers.

In certain embodiments, the ABT-263 salt, particle size, excipient ingredients and amounts thereof are selected to provide at least comparable bioabsorption by comparison with a standard solution of the drug, e.g., a solution in a carrier consisting of 10% DMSO in PEG-400, when administered orally. In other embodiments, the ABT-263 salt, particle size, excipient ingredients and amounts thereof are selected to provide enhanced bioabsorption by comparison with such a standard solution of the drug when administered orally. Comparable or enhanced bioabsorption can be evidenced by a pharmacokinetic (PK) profile having similar or higher Cₘₐₓ and/or similar or greater AUC, for example AUC₀₋₂₄ or AUC_{0-∞}. Illustratively, bioavailability can be expressed as a percentage, for example using the parameter F %, which computes AUC for oral delivery of a test composition as a percentage of AUC for intravenous (i.v.) delivery of the drug in a suitable solvent, taking into account any difference between oral and i.v. doses.

Bioavailability can be determined by PK studies in humans or in any suitable model species. For present purposes, a dog model, as illustratively described in Examples 1 and 2 below, is generally suitable. In various illustrative embodiments, compositions of the invention exhibit oral bioavailability of at least about 15%, at least about 20%, at least about 25% or at least about 30%, up to or exceeding about 50%, in a dog model, when administered as a single dose of about 2.5 to about 10 mg/kg to fasting or non-fasting animals.

The potential of the present invention to provide in a convenient oral dosage form a bioavailability that is at least comparable to that of a solution of ABT-263 in PEG-400/DMSO 10:1 described in above-cited U.S. Patent Application Publication No. 2007/0027135, is unexpected, especially in view of the fact that formulation changes apparently have little effect on bioavailability of earlier generations of Bcl-2 protein family inhibitors such as ABT-737. Bioavailability in a rat model of ABT-737 did not exceed about 6%, regardless of formulation.

The present invention is not limited by any process used to prepare a composition as embraced or described herein. Any suitable process of pharmacy can be used, including dry blending with or without direct compression, and wet or dry granulation. In the illustrative, non-limiting processes and compositions shown below, API can be used in unmilled form, e.g., with a D₉₀ particle size of about 20 to about 30 μm, or after milling to a desired size, e.g., pin-milled or jet-milled to a D₉₀ particle size of about 3 to about 10 μm.

An illustrative dry blending process is as follows. API (e.g., ABT-263 bis-HCl) is mixed with excipients except lubricant, for example by blending in a V-blender for approximately 20 minutes. Lubricant is then added. The resulting powder blend is compressed, for example at 500 lb, in a tablet press with suitable tooling to provide the size and shape of tablets desired. Alternatively, the powder blend is filled into capsules.

An illustrative composition prepared by the above process consists of the following ingredients (all percentages by weight):

| | | |
|---|---|---|
| ABT-263 bis-HCl | 10.75% | (10% free base equivalent) |
| silicified microcrystalline cellulose | 49.00% | |
| mannitol | 20.00% | |
| pregelatinized starch | 5.00% | |
| sodium starch glycolate | 10.00% | |
| poloxamer (Pluronic ™ F127) | 4.00% | |
| colloidal silicon dioxide | 1.00% | |
| sodium stearyl fumarate | 0.25% | |

Tablets of 50 mg ABT-263 dosage strength (total tablet weight 500 mg) are prepared from the above ingredients in a Carver press at 500 lb, with round tooling.

A first illustrative wet granulation process is as follows. API (e.g., ABT-263 bis-HCl) is suspended in a binder/surfactant solution (granulation liquid), then added to a blend of diluent(s) and disintegrant(s) in a food processor to prepare a granulate.

A second illustrative wet granulation process is as follows. API (e.g., ABT-263 bis-HCl) is mixed with excipients, including granulation liquid but excluding lubricant, and granulated in a food processor. The granules are dried and passed through a 20 mesh screen. Lubricant is then added.

A third illustrative wet granulation process is as follows. API (e.g., ABT-263 bis-HCl) is mixed with excipients, including granulation liquid and a first amount of disintegrant (intragranular excipients) but excluding lubricant, and granulated in a food processor. The granules are dried and passed through a 20 mesh screen. A second amount of disintegrant, lubricant and optionally other extragranular excipient(s) are then added.

Granules prepared by any of the above wet granulation processes can be compressed, for example at 500 lb, in a tablet press with suitable tooling to provide the size and shape of tablets desired. Alternatively, the granules can be filled into capsules.

A first illustrative tablet composition that can be prepared by any of the above wet granulation processes consists of the following ingredients (all percentages by weight):

| | | |
|---|---|---|
| ABT-263 bis-HCl | 10.75% | (10% free base equivalent) |
| microcrystalline cellulose | 83.50% | |
| povidone K-30 | 3.00% | |
| crospovidone | 1.50% | |
| poloxamer (Pluronic ™ F127) | 1.00% | |
| sodium stearyl fumarate | 0.25% | |

A second illustrative tablet composition that can be prepared by any of the above wet granulation processes consists of the following ingredients (all percentages by weight):

| | | |
|---|---|---|
| ABT-263 bis-HCl | 5.38% | (5% free base equivalent) |
| microcrystalline cellulose | 85.87% | |
| povidone K-30 | 3.00% | |
| crospovidone | 1.50% | |
| poloxamer (Pluronic ™ F127) | 4.00% | |
| sodium stearyl fumarate | 0.25% | |

A third illustrative tablet composition that can be prepared by any of the above wet granulation processes consists of the following ingredients (all percentages by weight):

| | | |
|---|---|---|
| ABT-263 bis-HCl | 10.75% | (10% free base equivalent) |
| microcrystalline cellulose | 50.00% | |
| mannitol | 20.00% | |
| povidone K-30 | 5.00% | |
| sodium starch glycolate | 10.00% | |
| poloxamer (Pluronic ™ F127) | 4.00% | |
| sodium stearyl fumarate | 0.25% | |

Tablets containing a 50 mg dose of ABT-263 are prepared from any of the above wet granulations.

An illustrative capsule composition that can be prepared by any of the above wet granulation processes consists of the following ingredients (all percentages by weight):

| | | |
|---|---|---|
| ABT-263 bis-HCl | 10.75% | (10% free base equivalent) |
| microcrystalline cellulose | 50.00% | |
| mannitol | 30.00% | |
| hydroxypropylcellulose | 3.00% | |
| sodium starch glycolate | 5.00% | |
| poloxamer (Pluronic ™ F127) | 1.00% | |
| sodium stearyl fumarate | 0.25% | |

The composition is filled into size 0 capsules.

Compositions embraced herein, including compositions described generally or with specificity herein, are useful for orally delivering ABT-263 to a subject. Accordingly, a method of the invention for delivering ABT-263 to a subject comprises orally administering a composition as described above.

The subject can be human or non-human (e.g., a farm, zoo, work or companion animal, or a laboratory animal used as a model) but in an important embodiment the subject is a human patient in need of the drug, for example to treat a disease characterized by apoptotic dysfunction and/or overexpression of an anti-apoptotic Bcl-2 family protein. A human subject can be male or female and of any age. The patient is typically an adult, but a method of the invention can be useful to treat a childhood cancer such as leukemia, for example acute lymphocytic leukemia, in a pediatric patient.

The composition is normally administered in an amount providing a therapeutically effective daily dose of the drug. The term “daily dose” herein means the amount of drug administered per day, regardless of the frequency of administration. For example, if the subject receives a unit dose of 150 mg twice daily, the daily dose is 300 mg. Use of the term “daily dose” will be understood not to imply that the specified dosage amount is necessarily administered once daily. However, in a particular embodiment the dosing frequency is once daily (q.d.), and the daily dose and unit dose are in this embodiment the same thing.

What constitutes a therapeutically effective dose depends on the bioavailability of the particular formulation, the subject (including species and body weight of the subject), the disease (e.g., the particular type of cancer) to be treated, the stage and/or severity of the disease, the individual subject's tolerance of the compound, whether the compound is administered in monotherapy or in combination with one or more other drugs, e.g., other chemotherapeutics for treatment of cancer, and other factors. Thus the daily dose can vary within wide margins, for example from about 10 to about 1,000 mg. Greater or lesser daily doses can be appropriate in specific situations. It will be understood that recitation herein of a “therapeutically effective” dose herein does not necessarily require that the drug be therapeutically effective if only a single such dose is administered; typically therapeutic efficacy depends on the composition being administered repeatedly according to a regimen involving appropriate frequency and duration of administration. It is strongly preferred that, while the daily dose selected is sufficient to provide benefit in terms of treating the cancer, it should not be sufficient to provoke an adverse side-effect to an unacceptable or intolerable degree. A suitable therapeutically effective dose can be selected by the physician of ordinary skill without undue experimentation based on the disclosure herein and on art cited herein, taking into account factors such as those mentioned above. The physician may, for example, start a cancer patient on a course of therapy with a relatively low daily dose and titrate the dose upwards over a period of days or weeks, to reduce risk of adverse side-effects.

Illustratively, suitable doses of ABT-263 are generally about 25 to about 1,000 mg/day, more typically about 50 to about 500 mg/day or about 200 to about 400 mg/day, for example about 50, about 100, about 150, about 200, about 250, about 300, about 350, about 400, about 450 or about 500 mg/day, administered at an average dosage interval of about 3 hours to about 7 days, for example about 8 hours to about 3 days, or about 12 hours to about 2 days. In most cases a once-daily (q.d.) administration regimen is suitable.

An “average dosage interval” herein is defined as a span of time, for example one day or one week, divided by the number of unit doses administered over that span of time. For example, where a drug is administered three times a day, around 8 am, around noon and around 6 pm, the average dosage interval is 8 hours (a 24-hour time span divided by 3). If the drug is formulated as a discrete dosage form such as a tablet or capsule, a plurality (e.g., 2 to 4) of dosage forms administered at one time is considered a unit dose for the purpose of defining the average dosage interval.

A daily dosage amount and dosage interval can, in some embodiments, be selected to maintain a plasma concentration of ABT-263 in a range of about 0.5 to about 10 μg/ml. Thus, during a course of ABT-263 therapy according to such embodiments, the steady-state peak plasma concentration (Cₘₐₓ) should in general not exceed about 10 μg/ml, and the steady-state trough plasma concentration (Cₘᵢₙ) should in general not fall below about 0.5 μg/ml. It will further be found desirable to select, within the ranges provided above, a daily dosage amount and average dosage interval effective to provide a Cₘₐₓ/Cₘᵢₙ ratio not greater than about 5, for example not greater than about 3, at steady-state. It will be understood that longer dosage intervals will tend to result in greater Cₘₐₓ/Cₘᵢₙ ratios. Illustratively, at steady-state, an ABT-263 Cₘₐₓ of about 3 to about 8 μg/ml and Cₘᵢₙ of about 1 to about 5 μg/ml can be targeted by the present method. Steady-state values of Cₘₐₓ and Cₘᵢₙ can be established in a human PK study, for example conducted according to standard protocols including but not limited to those acceptable to a regulatory agency such as the U.S. Food and Drug Administration (FDA).

One to a small plurality of tablets or capsules can be swallowed whole, typically with the aid of water or other imbibable liquid to help the swallowing process. Optionally, tablets may be broken before swallowing and can be scored to facilitate even breakage.

As compositions of the present invention are believed to exhibit only a minor food effect, administration according to the present embodiment can be with or without food, i.e., in a non-fasting or fasting condition. It is generally preferred to administer the present compositions to a non-fasting patient.

Compositions of the invention are suitable for use in monotherapy or in combination therapy, for example with other chemotherapeutics or with ionizing radiation. A particular advantage of the present invention is that it permits once-daily oral administration, a regimen which is convenient for the patient who is undergoing treatment with other orally administered drugs on a once-daily regimen. Oral administration is easily accomplished by the patient him/herself or by a caregiver in the patient's home; it is also a convenient route of administration for patients in a hospital or residential care setting.

Combination therapies illustratively include administration of a composition of the present invention comprising ABT-263 concomitantly with one or more of bortezomid, carboplatin, cisplatin, cyclophosphamide, dacarbazine, dexamethasone, docetaxel, doxorubicin, etoposide, fludarabine, hydroxydoxorubicin, irinotecan, paclitaxel, rapamycin, rituximab, vincristine and the like, for example with a polytherapy such as CHOP (cyclophosphamide+hydroxydoxorubicin+vincristine+prednisone), RCVP (rituximab+cyclophosphamide+vincristine+prednisone), R-CHOP (rituximab+CHOP) or DA-EPOCH-R (dose-adjusted etoposide, prednisone, vincristine, cyclophosphamide, doxorubicin and rituximab).

A composition of the invention, for example such a composition comprising ABT-263, can be administered in combination therapy with one or more therapeutic agents that include, but are not limited to, angiogenesis inhibitors, antiproliferative agents, other apoptosis promoters (for example, Bcl-xL, Bcl-w and Bfl-1 inhibitors), activators of a death receptor pathway, BiTE (bi-specific T-cell engager) antibodies, dual variable domain binding proteins (DVDs), inhibitors of apoptosis proteins (IAPs), microRNAs, mitogen-activated extracellular signal-regulated kinase inhibitors, multivalent binding proteins, poly-ADP (adenosine diphosphate)-ribose polymerase (PARP) inhibitors, small inhibitory ribonucleic acids (siRNAs), kinase inhibitors, receptor tyrosine kinase inhibitors, aurora kinase inhibitors, polo-like kinase inhibitors, bcr-abl kinase inhibitors, growth factor inhibitors, COX-2 inhibitors, non-steroidal anti-inflammatory drugs (NSAIDs), antimitotic agents, alkylating agents, antimetabolites, intercalating antibiotics, platinum-containing chemotherapeutic agents, growth factor inhibitors, ionizing radiation, cell cycle inhibitors, enzymes, topoisomerase inhibitors, biologic response modifiers, immunologicals, antibodies, hormonal therapies, retinoids, deltoids, plant alkaloids, proteasome inhibitors, HSP-90 inhibitors, histone deacetylase (HDAC) inhibitors, purine analogs, pyrimidine analogs, MEK inhibitors, CDK inhibitors, ErbB2 receptor inhibitors, mTOR inhibitors as well as other antitumor agents.

Angiogenesis inhibitors include, but are not limited to, EGFR inhibitors, PDGFR inhibitors, VEGFR inhibitors, TIE2 inhibitors, IGF1R inhibitors, matrix metalloproteinase 2 (MMP-2) inhibitors, matrix metalloproteinase 9 (MMP-9) inhibitors and thrombospondin analogs.

Examples of EGFR inhibitors include, but are not limited to, gefitinib, erlotinib, cetuximab, EMD-7200, ABX-EGF, HR3, IgA antibodies, TP-38 (IVAX), EGFR fusion protein, EGF-vaccine, anti-EGFR immunoliposomes and lapatinib.

Examples of PDGFR inhibitors include, but are not limited to, CP-673451 and CP-868596.

Examples of VEGFR inhibitors include, but are not limited to, bevacizumab, sunitinib, sorafenib, CP-547632, axitinib, vandetanib, AEE788, AZD-2171, VEGF trap, vatalanib, pegaptanib, IM862, pazopanib, ABT-869 and angiozyme.

Bcl-2 family protein inhibitors other than ABT-263 include, but are not limited to, AT-101 ((−)gossypol), Genasense™ Bcl-2-targeting antisense oligonucleotide (G3139 or oblimersen), IPI-194, IPI-565, ABT-737, GX-070 (obatoclax) and the like.

Activators of a death receptor pathway include, but are not limited to, TRAIL, antibodies or other agents that target death receptors (e.g., DR4 and DR5) such as apomab, conatumumab, ETR2-ST01, GDC0145 (lexatumumab), HGS-1029, LBY-135, PRO-1762 and trastuzumab.

Examples of thrombospondin analogs include, but are not limited to, TSP-1, ABT-510, ABT-567 and ABT-898.

Examples of aurora kinase inhibitors include, but are not limited to, VX-680, AZD-1152 and MLN-8054.

An example of a polo-like kinase inhibitor includes, but is not limited to, BI-2536.

Examples of bcr-abl kinase inhibitors include, but are not limited to, imatinib and dasatinib.

Examples of platinum-containing agents include, but are not limited to, cisplatin, carboplatin, eptaplatin, lobaplatin, nedaplatin, oxaliplatin and satraplatin.

Examples of mTOR inhibitors include, but are not limited to, CCl-779, rapamycin, temsirolimus, everolimus, RAD001 and AP-23573.

Examples of HSP-90 inhibitors include, but are not limited to, geldanamycin, radicicol, 17-AAG, KOS-953, 17-DMAG, CNF-101, CNF-1010,17-AAG-nab, NCS-683664, efungumab, CNF-2024, PU3, PU24FC1, VER-49009, IPI-504, SNX-2112 and STA-9090.

Examples of HDAC inhibitors include, but are not limited to, suberoylanilide hydroxamic acid (SAHA), MS-275, valproic acid, TSA, LAQ-824, trapoxin and depsipeptide.

Examples of MEK inhibitors include, but are not limited to, PD-325901, ARRY-142886, ARRY-438162 and PD-98059.

Examples of CDK inhibitors include, but are not limited to, flavopyridol, MCS-5A, CVT-2584, seliciclib ZK-304709, PHA-690509, BMI-1040, GPC-286199, BMS-387032, PD-332991 and AZD-5438.

Examples of COX-2 inhibitors include, but are not limited to, celecoxib, parecoxib, deracoxib, ABT-963, etoricoxib, lumiracoxib, BMS-347070, RS 57067, NS-398, valdecoxib, rofecoxib, SD-8381, 4-methyl-2-(3,4-dimethylphenyl)-1-(4-sulfamoylphenyl)-1H-pyrrole, T-614, JTE-522, S-2474, SVT-2016, CT-3 and SC-58125.

Examples of NSAIDs include, but are not limited to, salsalate, diflunisal, ibuprofen, ketoprofen, nabumetone, piroxicam, naproxen, diclofenac, indomethacin, sulindac, tolmetin, etodolac, ketorolac and oxaprozin.

Examples of ErbB2 receptor inhibitors include, but are not limited to, CP-724714, canertinib, trastuzumab, petuzumab, TAK-165, ionafamib, GW-282974, EKB-569, PI-166, dHER2, APC-8024, anti-HER/2 neu bispecific antibody B7.her2IgG3 and HER2 trifunctional bispecific antibodies mAB AR-209 and mAB 2B-1.

Examples of alkylating agents include, but are not limited to, nitrogen mustard N-oxide, cyclophosphamide, ifosfamide, trofosfamide, chlorambucil, melphalan, busulfan, mitobronitol, carboquone, thiotepa, ranimustine, nimustine, Cloretazine™ (laromustine), AMD-473, altretamine, AP-5280, apaziquone, brostallicin, bendamustine, carmustine, estramustine, fotemustine, glufosfamide, KW-2170, mafosfamide, mitolactol, lomustine, treosulfan, dacarbazine and temozolomide.

Examples of antimetabolites include, but are not limited to, methotrexate, 6-mercaptopurine riboside, mercaptopurine, 5-fluorouracil (5-FU) alone or in combination with leucovorin, tegafur, UFT, doxifluridine, carmofur, cytarabine, cytarabine ocfosfate, enocitabine, S-1, pemetrexed, gemcitabine, fludarabine, 5-azacitidine, capecitabine, cladribine, clofarabine, decitabine, eflornithine, ethenylcytidine, cytosine arabinoside, hydroxyurea, TS-1, melphalan, nelarabine, nolatrexed, disodium pemetrexed, pentostatin, pelitrexol, raltitrexed, triapine, trimetrexate, vidarabine, mycophenolic acid, ocfosfate, pentostatin, tiazofurin, ribavirin, EICAR, hydroxyurea and deferoxamine.

Examples of antibiotics include, but are not limited to, intercalating antibiotics, aclarubicin, actinomycin D, amrubicin, annamycin, adriamycin, bleomycin, daunorubicin, doxorubicin (including liposomal doxorubicin), elsamitrucin, epirubicin, glarubicin, idarubicin, mitomycin C, nemorubicin, neocarzinostatin, peplomycin, pirarubicin, rebeccamycin, stimalamer, streptozocin, valrubicin, zinostatin and combinations thereof.

Examples of topoisomerase inhibiting agents include, but are not limited to, aclarubicin, amonafide, belotecan, camptothecin, 10-hydroxycamptothecin, 9-amino-camptothecin, amsacrine, dexrazoxane, diflomotecan, irinotecan HCl, edotecarin, epirubicin, etoposide, exatecan, becatecarin, gimatecan, lurtotecan, orathecin, BN-80915, mitoxantrone, pirarbucin, pixantrone, rubitecan, sobuzoxane, SN-38, tafluposide and topotecan.

Examples of antibodies include, but are not limited to, rituximab, cetuximab, bevacizumab, trastuzumab, CD40-specific antibodies and IGF1R-specific antibodies, chTNT-1/B, denosumab, edrecolomab, WX G250, zanolimumab, lintuzumab and ticilimumab.

Examples of hormonal therapies include, but are not limited to, sevelamer carbonate, rilostane, luteinizing hormone releasing hormone, modrastane, exemestane, leuprolide acetate, buserelin, cetrorelix, deslorelin, histrelin, anastrozole, fosrelin, goserelin, degarelix, doxercalciferol, fadrozole, formestane, tamoxifen, arzoxifene, bicalutamide, abarelix, triptorelin, finasteride, fulvestrant, toremifene, raloxifene, trilostane, lasofoxifene, letrozole, flutamide, megesterol, mifepristone, nilutamide, dexamethasone, prednisone and other glucocorticoids.

Examples of retinoids or deltoids include, but are not limited to, seocalcitol, lexacalcitol, fenretinide, aliretinoin, tretinoin, bexarotene and LGD-1550.

Examples of plant alkaloids include, but are not limited to, vincristine, vinblastine, vindesine and vinorelbine.

Examples of proteasome inhibitors include, but are not limited to, bortezomib, MG-132, NPI-0052 and PR-171.

Examples of immunologicals include, but are not limited to, interferons and numerous other immune-enhancing agents. Interferons include interferon alpha, interferon alpha-2a, interferon alpha-2b, interferon beta, interferon gamma-1a, interferon gamma-1b, interferon gamma-n1 and combinations thereof. Other agents include filgrastim, lentinan, sizofilan, BCG live, ubenimex, WF-10 (tetrachlorodecaoxide or TCDO), aldesleukin, alemtuzumab, BAM-002, dacarbazine, daclizumab, denileukin, gemtuzumab ozogamicin, ibritumomab, imiquimod, lenograstim, melanoma vaccine, molgramostim, sargaramostim, tasonermin, tecleukin, thymalasin, tositumomab, Virulizin™ immunotherapeutic of Lorus Pharmaceuticals, Z-100 (specific substance of Maruyama or SSM), Zevalin™ (90Y-ibritumomab tiuxetan), epratuzumab, mitumomab, oregovomab, pemtumomab, Provenge™ (sipuleucel-T), teceleukin, Therocys™ (Bacillus Calmette-Guerin), cytotoxic lymphocyte antigen 4 (CTLA4) antibodies and agents capable of blocking CTLA4 such as MDX-010.

Examples of biological response modifiers are agents that modify defense mechanisms of living organisms or biological responses, such as survival, growth, or differentiation of tissue cells to direct them to have anti-tumor activity. Such agents include, but are not limited to, krestin, lentinan, sizofuran, picibanil, PF-3512676 and ubenimex.

Examples of pyrimidine analogs include, but are not limited to, 5-fluorouracil, floxuridine, doxifluridine, raltitrexed, cytarabine, cytosine arabinoside, fludarabine, triacetyluridine, troxacitabine and gemcitabine.

Examples of purine analogs include, but are not limited to, mercaptopurine and thioguanine.

Examples of antimitotic agents include, but are not limited to, N-(2-((4-hydroxyphenyl)amino)pyridin-3-yl)-4-methoxybenzenesulfonamide, paclitaxel, docetaxel, larotaxel, epothilone D, PNU-100940, batabulin, ixabepilone, patupilone, XRP-9881, vinflunine and ZK-EPO (synthetic epothilone).

Examples of radiotherapy include, but are not limited to, external beam radiotherapy (XBRT), teletherapy, brachytherapy, sealed-source radiotherapy and unsealed-source radiotherapy.

BiTE antibodies are bi-specific antibodies that direct T-cells to attack cancer cells by simultaneously binding the two cells. The T-cell then attacks the target cancer cell. Examples of BiTE antibodies include, but are not limited to, adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (Sutton et al. (1997) J. Immunol. 158:5783-5790).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand. For example, siRNAs targeting Mcl-1 have been shown to enhance the activity of ABT-263 (Tse et al. (2008) Cancer Res. 68:3421-3428 and references therein).

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy-chain DVD polypeptides and two light-chain DVD polypeptides are referred to as DVD Ig's. Each half of a DVD Ig comprises a heavy-chain DVD polypeptide, a light-chain DVD polypeptide, and two antigen binding sites. Each binding site comprises a heavy-chain variable domain and a light-chain variable domain with a total of 6 CDRs involved in antigen binding per antigen binding site.

PARP inhibitors include, but are not limited to, ABT-888, olaparib, KU-59436, AZD-2281, AG-014699, BSI-201, BGP-15, INO-1001, ONO-2231 and the like.

Additionally or alternatively, a composition of the present invention can be administered in combination therapy with one or more antitumor agents selected from ABT-100, N-acetylcolchinol-O-phosphate, acitretin, AE-941, aglycon protopanaxadiol, arglabin, arsenic trioxide, AS04 adjuvant-adsorbed HPV vaccine, L-asparaginase, atamestane, atrasentan, AVE-8062, bosentan, canfosfamide, Canvaxin™, catumaxomab, CeaVac™ celmoleukin, combrestatin A4P, contusugene ladenovec, Cotara™, cyproterone, deoxycoformycin, dexrazoxane, N,N-diethyl-2-(4-(phenylmethyl)phenoxy)ethanamine, 5,6-dimethylxanthenone-4-acetic acid, docosahexaenoic acid/paclitaxel, discodermolide, efaproxiral, enzastaurin, epothilone B, ethynyluracil, exisulind, falimarev, Gastrimmune™ GMK vaccine, GVAX™, halofuginone, histamine, hydroxycarbamide, ibandronic acid, ibritumomab tiuxetan, IL-13-PE38, inalimarev, interleukin 4, KSB-311, lanreotide, lenalidomide, lonafarnib, lovastatin, 5,10-methylenetetrahydrofolate, mifamurtide, miltefosine, motexafin, oblimersen, OncoVAX™, Osidem™, paclitaxel albumin-stabilized nanoparticle, paclitaxel poliglumex, pamidronate, panitumumab, peginterferon alfa, pegaspargase, phenoxodiol, poly(I)-poly(C12U), procarbazine, ranpirnase, rebimastat, recombinant quadrivalent HPV vaccine, squalamine, staurosporine, STn-KLH vaccine, T4 endonuclase V, tazarotene, 6,6′,7,12-tetramethoxy-2,2′-dimethyl-1β-berbaman, thalidomide, TNFerade™, ¹³¹I-tositumomab, trabectedin, triazone, tumor necrosis factor, Ukrain™, vaccinia-MUC-1 vaccine, L-valine-L-boroproline, Vitaxin™, vitespen, zoledronic acid and zorubicin.

In one embodiment, a composition of the invention comprising ABT-263 is administered in a therapeutically effective amount to a subject in need thereof to treat a disease during which is overexpressed one or more of antiapoptotic Bcl-2 protein, antiapoptotic Bcl-X_{L} protein and antiapoptotic Bcl-w protein.

In another embodiment, a composition of the invention comprising ABT-263 is administered in a therapeutically effective amount to a subject in need thereof to treat a disease of abnormal cell growth and/or dysregulated apoptosis.

Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In a more particular embodiment, a composition of the invention comprising ABT-263 is administered in a therapeutically effective amount to a subject in need thereof to treat bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer or spleen cancer.

According to any of these embodiments, the composition is administered in monotherapy or in combination therapy with one or more additional therapeutic agents.

For example, a method for treating mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof in a subject comprises administering to the subject therapeutically effective amounts of (a) a composition of the invention, for example such a composition comprising ABT-263, and (b) one or more of etoposide, vincristine, CHOP, rituximab, rapamycin, R-CHOP, RCVP, DA-EPOCH-R or bortezomib.

In particular embodiments, a composition of the invention comprising ABT-263 is administered in a therapeutically effective amount to a subject in need thereof in monotherapy or in combination therapy with etoposide, vincristine, CHOP, rituximab, rapamycin, R-CHOP, RCVP, DA-EPOCH-R or bortezomib in a therapeutically effective amount, for treatment of a lymphoid malignancy such as B-cell lymphoma or non-Hodgkin's lymphoma.

In other particular embodiments, a composition of the invention comprising ABT-263 is administered in a therapeutically effective amount to a subject in need thereof in monotherapy or in combination therapy with etoposide, vincristine, CHOP, rituximab, rapamycin, R-CHOP, RCVP, DA-EPOCH-R or bortezomib in a therapeutically effective amount, for treatment of chronic lymphocytic leukemia or acute lymphocytic leukemia.

The present invention also provides a method for maintaining in bloodstream of a human cancer patient a therapeutically effective plasma concentration of ABT-263 and/or one or more metabolites thereof, comprising administering to the subject a pharmaceutical composition as described herein, in a dosage amount equivalent to about 50 to about 500 mg ABT-263 per day, at an average dosage interval of about 3 hours to about 7 days.

What constitutes a therapeutically effective plasma concentration depends inter alfa on the particular cancer present in the patient, the stage, severity and aggressiveness of the cancer, and the outcome sought (e.g., stabilization, reduction in tumor growth, tumor shrinkage, reduced risk of metastasis, etc.). It is strongly preferred that, while the plasma concentration is sufficient to provide benefit in terms of treating the cancer, it should not be sufficient to provoke an adverse side-effect to an unacceptable or intolerable degree.

For treatment of cancer in general and of a lymphoid malignancy such as non-Hodgkin's lymphoma in particular, the plasma concentration of ABT-263 should in most cases be maintained in a range of about 0.5 to about 10 μg/ml. Thus, during a course of ABT-263 therapy, the steady-state Cₘₐₓ should in general not exceed about 10 μg/ml, and the steady-state Cₘᵢₙ, should in general not fall below about 0.5 μg/ml. It will further be found desirable to select, within the ranges provided above, a daily dosage amount and average dosage interval effective to provide a Cₘₐₓ/Cₘᵢₙ, ratio not greater than about 5, for example not greater than about 3, at steady-state. It will be understood that longer dosage intervals will tend to result in greater Cₘₐₓ/Cₘᵢₙ, ratios. Illustratively, at steady-state, an ABT-263 Cₘₐₓ of about 3 to about 8 μg/ml and Cₘᵢₙ, of about 1 to about 5 μg/ml can be targeted by the present method.

A daily dosage amount effective to maintain a therapeutically effective ABT-263 plasma level is, according to the present embodiment, about 50 to about 500 mg. In most cases a suitable daily dosage amount is about 200 to about 400 mg. Illustratively, the daily dosage amount can be for example about 50, about 100, about 150, about 200, about 250, about 300, about 350, about 400, about 450 or about 500 mg.

An average dosage interval effective to maintain a therapeutically effective ABT-263 plasma level is, according to the present embodiment, about 3 hours to about 7 days. In most cases a suitable average dosage interval is about 8 hours to about 3 days, or about 12 hours to about 2 days. A once-daily (q.d.) administration regimen is often suitable.

As in other embodiments, administration according to the present embodiment can be with or without food, i.e., in a non-fasting or fasting condition. It is generally preferred to administer the present compositions to a non-fasting patient.

Further information of relevance to the present invention is available in a recently published article by Tse et al. (2008) Cancer Res. 68:3421-3428 and supplementary data thereto available at Cancer Research Online (cancerres.aacrjournals.org/). This article and its supplementary data are incorporated in their entirety herein by reference.

### EXAMPLES

The following examples are merely illustrative, and do not limit this disclosure in any way. Trademarked ingredients used in the examples can be substituted with comparable ingredients from other suppliers. Trademarked ingredients used in the examples include:

Avicel 101™ and Avicel 102™ of FMC: microcrystalline cellulose;

Cremophor EL™ of BASF: polyoxyl 35 castor oil;

ProSolv HD 90™ of JRS Pharma: silicified microcrystalline cellulose;

Starch 1500™ of Colorcon: pregelatinized starch.

### Example 1

### PK Studies of ABT-263 Solid Tablets in Dogs

PK studies were performed in non-fasting beagle dogs (n=3) at a single dose of 50 mg ABT-263 free base equivalent. Plasma concentrations of the drug were determined by high pressure liquid chromatography mass spectrometry (HPLC-MS) and PK parameters were calculated by standard procedures in the art.

Eleven tablet compositions of the invention (Formulations A-K) were tested. API (ABT-263 bis-HCl in all cases) was unmilled unless otherwise indicated. Composition of each of Formulations A-E is as shown in Table 1.

TABLE 1
Composition of tablets (Formulations A-E)

| | Amount (% by weight) | | | | |
|---|---|---|---|---|---|
| Ingredient | A | B | C | D | E |
| ABT-263 bis-HCl | 10.00 | 10.00 | 10.00 | 10.75 | 10.75 |
| Avicel 101 ™ | 81.25 | 84.25 | 50.75 | 30.00 | 30.00 |
| mannitol | | | 20.00 | 40.00 | 40.00 |
| PVP K-30 | 3.00 | 3.00 | 5.00 | 5.00 | 3.00 |
| crospovidone | 1.50 | 1.50 | | | |
| poloxamer (Pluronic ™ F127) | 4.00 | 1.00 | 4.00 | | |
| TPGS | | | | 4.00 | 6.00 |
| sodium starch glycolate | | | 10.00 | 10.00 | 10.00 |
| magnesium stearate | 0.25 | 0.25 | 0.25 | 0.25 | 0.25 |

Formulations F-K comprised intra- and extragranular components. Composition of each of these formulations is as shown in Table 2.

TABLE 2
Composition of tablets (Formulations F-K)

| | Amount (% by weight) | | | | | |
|---|---|---|---|---|---|---|
| Ingredient | F | G | H | I | J | K |

Intragranular

| | | | | | | |
|---|---|---|---|---|---|---|
| ABT-263 bis-HCl | 10.75 | 10.75 | 10.75 | 21.50 | 10.75 | 21.50 |
| Avicel 101 ™ | 33.00 | 34.00 | 30.00 | 29.25 | 30.00 | 29.25 |
| mannitol | 20.00 | 20.00 | 20.00 | 20.00 | 30.00 | 20.00 |
| PVP 30 | 5.00 | 5.00 | 5.00 | 5.00 | 5.00 | 5.00 |
| poloxamer (Pluronic ™ F127) | 1.00 | | | | | |
| sodium starch glycolate | | | 5.00 | 5.00 | 5.00 | 5.00 |
| Cremophor EL ™ | | | 4.00 | 4.00 | | |
| TPGS | | | | | 4.00 | 4.00 |

Extragranular

| | | | | | | |
|---|---|---|---|---|---|---|
| Avicel 101 ™ | 20.00 | 20.00 | 20.00 | 10.00 | 20.00 | 20.00 |
| sodium starch glycolate | 5.00 | 5.00 | 5.00 | 5.00 | 5.00 | 5.00 |
| magnesium stearate | 0.25 | 0.25 | 0.25 | 0.25 | 0.25 | 0.25 |

Formulation L consists of the following ingredients (all percentages by weight):

| | |
|---|---|
| ABT-263 bis-HCl | 10.75% |
| ProSolv HD 90 ™ | 49.00% |
| mannitol | 20.00% |
| Starch 1500 ™ | 5.00% |
| sodium starch glycolate | 10.00% |
| poloxamer (Pluronic ™ F127) | 4.00% |
| colloidal silicon dioxide | 1.00% |
| sodium stearyl fumarate | 0.25% |

Tablets were prepared by one of the processes shown in Table 3.

TABLE 3
Processes used in preparing tablets

| Process | Description |
|---|---|
| I | Wet granulation; API suspended in binder solution |
| | (PVP + poloxamer) |
| II | Wet granulation; API blended intragranularly |
| III | Dry blend; directly compressed tablets |

Table 4 summarizes PK data for ABT-263 tablet formulations in dogs. F % is a measure of bioavailability.

TABLE 4
PK data for tablet formulations

| | | | | AUC | |
|---|---|---|---|---|---|
| Formulation | Process | Tₘₐₓ (h) | Cₘₐₓ (μg/ml) | (μg · h/ml) | F % |
| A | I | 5.3 ± 1.2 | 2.2 ± 1.0 | 24.1 | 9.6 |
| | I | 2.3 ± 0.6 | 3.5 ± 0.3 | 28.5 | 12.0 |
| | API | | | | |
| | jet-milled | | | | |
| B | I | 7.0 ± 6.9 | 1.8 ± 0.5 | 20.1 | 8.3 |
| | II | 3.0 ± 0.0 | 4.0 ± 1.1 | 37.7 | 16.8 |
| C | I | 7.3 ± 6.7 | 3.6 ± 1.6 | 47.7 | 21.5 |
| D | II | 6.7 ± 5.0 | 3.9 ± 2.2 | 37.5 | 14.9 |
| E | II | 1.8 ± 0.3 | 7.5 ± 2.3 | 60.7 | 22.6 |
| F | II | 2.7 ± 0.6 | 6.1 ± 2.5 | 47.6 | 20.6 |
| G | II | 2.3 ± 0.6 | 7.1 ± 3.2 | 42.6 | 18.6 |
| H | II | 4.3 ± 4.0 | 3.6 ± 1.1 | 34.5 | 13.6 |
| I | II | 3.7 ± 2.1 | 5.8 ± 1.5 | 48.3 | 19.2 |
| J | II | 3.0 ± 1.0 | 6.8 ± 1.3 | 69.9 | 25.5 |
| K | II | 3.0 ± 1.0 | 4.5 ± 3.2 | 51.7 | 20.4 |
| L | III | 3.0 ± 1.0 | 10.2 ± 2.9 | 76.2 | 31.0 |

Tablets prepared by direct compression (Process III) exhibited higher bioavailability in these dog studies than those prepared by wet granulation (Processes I and II). Tablets prepared by Process II generally provided higher bioavailability in dogs than those prepared by Process I. Adding the drug by suspending it in the binder solution also appeared to prolong the Tₘₐₓ.

Addition of a surfactant to tablets made by wet granulation did not significantly change in vivo absorption of the drug. Addition of water-soluble excipients such as mannitol appeared to enhance in vivo drug absorption.

A change in drug loading level (21.5% vs. 10.75% ABT-263 bis-HCl; 20% vs. 10% free base equivalent)) did not significantly change bioavailability.

Increasing the binder (e.g., PVP) concentration for wet granulation had a tendency to reduce bioavailability.

### Example 2

### PK Studies of ABT-263 Solid Capsules in Dogs

PK studies were performed in non-fasting beagle dogs (n=3) at a single dose of 50 mg ABT-263 free base equivalent. Plasma concentrations of the drug were determined by high pressure liquid chromatography mass spectrometry (HPLC-MS) and PK parameters were calculated by standard procedures in the art.

Four capsule compositions of the invention (containing Formulations M-P) were tested. API (ABT-263 bis-HCl in all cases) was unmilled unless otherwise indicated.

Formulation M consists of the following ingredients (all percentages by weight):

| | |
|---|---|
| ABT-263 bis-HCl | 10.75% |
| ProSolv HD 90 ™ | 49.00% |
| mannitol | 20.00% |
| starch 1500 | 5.00% |
| sodium starch glycolate | 10.00% |
| poloxamer (Pluronic ™ F127) | 4.00% |
| colloidal silicon dioxide | 1.00% |
| magnesium stearate | 0.25% |

Formulation N consists of an intragranular component and an extragranular component having the following ingredients (all percentages by weight):

Intragranular

| | |
|---|---|
| ABT-263 bis-HCl | 10.75% |
| Avicel 101 ™ | 30.00% |
| mannitol | 30.00% |
| poloxamer (Pluronic ™ F127) | 1.00% |
| hydroxypropylcellulose | 3.00% |
| sodium starch glycolate | 2.5% |

Extragranular

| | |
|---|---|
| Avicel 101 ™ | 20.00% |
| sodium starch glycolate | 2.5% |
| sodium stearyl fumarate | 0.25% |

Formulation 0 consists of the following ingredients (all percentages by weight):

| | |
|---|---|
| ABT-263 bis-HCl | 10.75% |
| ProSolv HD 90 ™ | 50.00% |
| mannitol | 30.00% |
| hydroxypropylcellulose | 3.00% |
| poloxamer (Pluronic ™ F127) | 1.00% |
| sodium starch glycolate | 5.00% |
| sodium stearyl fumarate | 0.25% |

Formulation P consists of the following ingredients (all percentages by weight):

| | |
|---|---|
| ABT-263 bis-HCl | 16.12% |
| Avicel 102 ™ | 50.00% |
| mannitol | 28.13% |
| sodium starch glycolate | 5.00% |
| colloidal silicon dioxide | 0.50% |
| sodium stearyl fumarate | 0.25% |

Capsule fills were prepared by one of the processes shown in Table 5.

TABLE 5
Processes used in preparing capsules

| Process | Description |
|---|---|
| II | Wet granulation; API blended intragranularly |
| IV | Dry blend encapsulation |

Table 6 summarizes PK data for ABT-263 tablet formulations in dogs. Formulation P was tested three times.

TABLE 6
PK data for capsule formulations

| | | | | AUC | |
|---|---|---|---|---|---|
| Formulation | Process | Tₘₐₓ (h) | Cₘₐₓ (μg/ml) | (μg · h/ml) | F % |
| M | IV | 4.2 ± 2.4 | 6.3 ± 1.5 | 54.1 | 21.7 |
| N | II | 6.7 ± 5.4 | 4.7 ± 2.4 | 51.0 | 20.3 |
| | II | 3.8 ± 1.3 | 45 ± 1.9 | 40.5 | 13.2 |
| | API | | | | |
| | jet-milled | | | | |
| O | III | 3.2 ± 0.8 | 6.2 ± 2.0 | 53.0 | 21.0 |
| | III | 4.7 ± 3.7 | 7.4 ± 2.0 | 74.5 | 34.2 |
| | API | | | | |
| | jet-milled | | | | |
| P | IV | 2.8 ± 0.7 | 2.5 ± 0.5 | 43.2 | 15.8 |
| | | 7.0 ± 4.8 | 5.0 ± 1.2 | 62.3 | 23.5 |
| | | 4.2 ± 1.5 | 6.4 ± 2.9 | 52.6 | 17.6 |

Micronization of the API by jet-milling led to improved bioavailability for capsules made by dry blending (Process IV) but not by wet granulation (Process II). Addition of poloxamer surfactant did not significantly affect bioavailability of a dry blend encapsulation formulation.

What is claimed is:

1. An orally deliverable pharmaceutical composition comprising in solid-state form (a) a pharmaceutically acceptable acid addition salt of ABT-263 (N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino-3-((trifluoromethyl)sulfonyl)benzene-sulfonamide), and (b) a plurality of pharmaceutically acceptable excipients including at least a solid diluent and a solid disintegrant; wherein the salt is formed from more than one equivalent of acid per equivalent of ABT-263.

2. The composition of claim 1, in a unit dosage form.

3. The composition of claim 2, wherein the unit dosage form is a tablet or capsule.

4. The composition of claim 1, wherein the salt of ABT-263 is a bis-acid addition salt.

5. The composition of claim 1, wherein the salt of ABT-263 is ABT-263 bis-HCl.

6. The composition of claim 1, wherein the ABT-263 salt is present in an amount of about 2% to about 40% free base equivalent by weight.

7. The composition of claim 1, wherein the ABT-263 salt has a D₉₀ particle size of about 2.5 to about 50 μm.

8. The composition of claim 1, comprising about 5% to about 95% by weight in total of one or more diluents.

9. The composition of claim 1, comprising one or more diluents selected from the group consisting of lactose, anhydrous lactose, lactose monohydrate, lactitol, maltitol, mannitol, sorbitol, xylitol, dextrose, dextrose monohydrate, fructose, sucrose, compressible sugar, confectioner's sugar, sugar spheres, maltose, inositol, hydrolyzed cereal solids, starch, amylose, dextrates, pregelatinized starch, dextrins, powdered cellulose, microcrystalline cellulose, silicified microcrystalline cellulose, food-grade sources of β- and amorphous cellulose and powdered cellulose, cellulose acetate, calcium carbonate, tribasic calcium phosphate, dibasic calcium phosphate dihydrate, monobasic calcium sulfate monohydrate, calcium sulfate, granular calcium lactate trihydrate, magnesium carbonate, magnesium oxide, bentonite, kaolin and sodium chloride.

10. The composition of claim 1, comprising as diluent microcrystalline cellulose, silicified microcrystalline cellulose or a combination thereof.

11. The composition of claim 10, further comprising a water-soluble diluent.

12. The composition of claim 11, wherein the water-soluble diluent comprises mannitol.

13. The composition of claim 1, comprising about 0.2% to about 30% by weight in total of one or more disintegrants.

14. The composition of claim 1, comprising one or more disintegrants selected from the group consisting of pregelatinized starch, sodium starch glycolate, clays, magnesium aluminum silicate, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums and colloidal silicon dioxide.

15. The composition of claim 1, comprising as disintegrant sodium starch glycolate.

16. The composition of claim 1, comprising granules having intragranular and/or extragranular disintegrant.

17. The composition of claim 1, further comprising about 0.5% to about 25% by weight in total of one or more binding agents.

18. The composition of claim 1, further comprising one or more binding agents selected from the group consisting of acacia, tragacanth, glucose, polydextrose, starch, pregelatinized starch, gelatin, methylcellulose, carmellose sodium, HPMC, hydroxypropylcellulose, hydroxyethylcellulose, ethylcellulose, dextrins, maltodextrin, zein, alginic acid, sodium alginate, magnesium aluminum silicate, bentonite, PEG, polyethylene oxide, guar gum, polysaccharide acids, povidone, carbomers and polymethacrylates.

19. The composition of claim 1, further comprising as binding agent povidone, HPMC or a combination thereof.

20. The composition of claim 1, further comprising about 0.1% to about 15% by weight in total of one or more wetting agents.

21. The composition of claim 1, further comprising one or more wetting agents selected from the group consisting of benzalkonium chloride, benzethonium chloride, cetylpyridinium chloride, dioctyl sodium sulfosuccinate, polyoxyethylene alkylphenyl ethers, poloxamers, polyoxyethylene fatty acid glycerides and oils, polyoxyethylene (8) caprylic/capric mono- and diglycerides, polyoxyethylene (35) castor oil, polyoxyethylene (40) hydrogenated castor oil, polyoxyethylene alkyl ethers, ceteth-10, laureth-4, laureth-23, oleth-2, oleth-10, oleth-20, steareth-2, steareth-10, steareth-20, steareth-100, polyoxyethylene (20) cetostearyl ether, polyoxyethylene fatty acid esters, polyoxyethylene (20) stearate, polyoxyethylene (40) stearate, polyoxyethylene (100) stearate, sorbitan esters, sorbitan monolaurate, sorbitan monooleate, sorbitan monopalmitate, sorbitan monostearate, polyoxyethylene sorbitan esters, polysorbate 20, polysorbate 80, propylene glycol fatty acid esters, propylene glycol laurate, sodium lauryl sulfate, fatty acids and salts thereof, oleic acid, sodium oleate, triethanolamine oleate, glyceryl fatty acid esters, glyceryl monooleate, glyceryl monostearate, glyceryl palmitostearate, TPGS and tyloxapol.

22. The composition of claim 1, further comprising as wetting agent a poloxamer.

23. The composition of claim 1, further comprising about 0.05% to about 10% in total of one or more lubricants.

24. The composition of claim 1, further comprising one or more lubricants selected from the group consisting of glyceryl behenate, stearic acid, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEG, poloxamers, polyvinyl alcohol, sodium oleate, sodium lauryl sulfate and magnesium lauryl sulfate.

25. The composition of claim 1, further comprising as lubricant sodium stearyl fumarate.

26. A method for treating a disease characterized by apoptotic dysfunction and/or overexpression of an anti-apoptotic Bcl-2 family protein, comprising orally administering to a subject having the disease a therapeutically effective amount of the composition of claim 1.

27. The method of claim 26, wherein the disease is a neoplastic disease.

28. The method of claim 27, wherein the neoplastic disease is selected from the group consisting of cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma and combinations thereof.

29. The method of claim 27, wherein the neoplastic disease is a lymphoid malignancy.

30. The method of claim 29, wherein the lymphoid malignancy is non-Hodgkin's lymphoma.

31. The method of claim 27, wherein the neoplastic disease is chronic lymphocytic leukemia or acute lymphocytic leukemia.

32. The method of claim 26, wherein the composition administered comprises ABT-263 bis-HCl.

33. The method of claim 26, wherein the composition is administered in a dose of about 50 to about 500 mg ABT-263 free base equivalent per day at an average treatment interval of about 3 hours to about 7 days.

34. The method of claim 26, wherein the composition is administered once daily in a dose of about 200 to about 400 mg ABT-263 free base equivalent per day.
